(19) United States
(12) Patent Application Publication
(10) Pub. No.: US 2001/0034056 A1
(43) Pub. Date: Oct. 25, 2001

(54) Odor control scrubber

(76) Inventor: Kenneth J. Corey, Yucaipa, CA (US)

Correspondence Address: Stephen R. Seccombe, Sheldon & Mak, Suite 503, 290 North 'D' Street, San Bernardino, CA, 92401, US

(21) Appl. No.: 09/800,419

(22) Filed: Mar. 5, 2001

Related U.S. Application Data
(60) Provisional application No. 60/186,899, filed on Mar. 3, 2000.

(51) Int. Cl. C12S 5/00; C12M 1/04

(52) U.S. Cl. 435/266; 435/299.1; 435/309.2

(57) ABSTRACT

A counterflow scrubbing system for deodorizing air having sulfur components such as H₂S typically associated with wastewater treatment includes a tower vessel having sulfer-oxidizing microorganisms in porous rock media, and operates at a pH preferably between 1.5 and 4.0 The media has a high ratio of surface area to volume, being at least 1000 and preferably approximately 10,000. The system can operate continuously without requiring objectionable chemicals, relaying of filter beds, or back-flushing. Optionally, concentrations of nutrients and/or bacteria are added to make-up water from an included reservoir.

### CROSS-REFERENCE TO RELATED APPLICATIONS

This application claims the benefit of Provisional Patent Application Ser. No. 60/186,899, filed Mar. 3, 2000, the contents of which are incorporated herein by this reference.

### BACKGROUND

The present invention is related to wastewater treatment, and more particularly to the control of offensive odors that are typically associated with such treatment.

A typical and major problem in wastewater treatment plants is controlling the emissions of odors to a level that does not create a public nuisance. The financial costs related to odor control can be significant. In addition, if the odor treatment methods are not effective, the plant can experience loss of good will in the community, and in some instances fines or penalties. The principal odorous compound in a wastewater treatment plant is usually hydrogen sulfide (H₂S). Historically the elimination of this odor has involved the use of chemical or physical treatment technologies, such as wet chemical scrubbers and activated carbon adsorbers. The magnitude of this problem stems from the significant concentrations of hydrogen sulfide in wastewater treatment offgases, and the consequent overwhelming contribution to the odors associated with wastewater treatment.

Recently, research has been conducted on biological treatment methods for controlling odors, for reducing the cost of such treatment, and for avoiding the introduction of other unwanted chemicals to the air stream. Thus the use of microorganisms to remove odors at wastewater plants is not new. In fact, solid matrix biofiltration has been used for many years in other countries, and in recent years has become more common within the United States. It has been demonstrated that low cost removal of H₂S can easily be accomplished by biological treatment. However, biofilters require large land areas, and there is eventual loss of treatment efficiency because of compaction, and channeling of the solid organic media. Further, because of the high concentrations of H₂S in the offgases from most covered processes, the solid-phase biofilter eventually breaks down due to the effects of the acid produced in the media by microorganisms biodegrading the H₂S. In addition to these drawbacks, sulfur accumulation in the bed, from partial oxidation of H₂S, can also affect performance by coating the media and increasing the head losses through the bed. These problems have discourage the use of biological agents in wastewater treatment.

Thus there is a need for a biological odor elimination system that effectively controls odors of H₂S and other sulfur compounds, and that overcomes the disadvantages of the prior art.

### SUMMARY

The present invention meets this need by providing a system that effectively eliminates odors of H₂S and other airborne pollutants associated with wastewater treatment. In one aspect of the invention, a gas-liquid scrubber system includes a tower vessel having a gas inlet for receiving a gas stream and an exhaust outlet, a perforate media support between the gas inlet and the gas outlet for supporting porous media with the gas stream passing therethrough, and a sump for collecting liquid falling below the media support; a liquid recirculation system having a pump fluid connected to the sump, a nozzle in the tower vessel, and a conduit connected between the pump and the nozzle for spraying the media with the liquid when the media is supported on the media support structure and a quantity of the liquid is present in the sump, the liquid also passing through the media to the sump; means for populating the media with sulfur-oxidizing microorganisms; and means for maintaining a pH of the recirculating liquid between a low limit and a high limit, the low limit being not less than 1.0, the high limit being not greater than 5.0. The means for populating the media can include a fill conduit for receiving fill water containing the microorganisms into the recirculation system. The means for populating the media can also include an inlet conduit for receiving make-up water, a reservoir containing a concentration of the microorganisms, and a feeder connected between the inlet conduit, the reservoir, and the fill conduit for mixing a dosage of the concentration of microorganisms with the make-up water to produce the fill water. The microorganisms can include thiobacillus bacteria. Alternatively, the means for populating the media can include an access structure formed in the tower vessel for admitting a concentration of the microorganisms into the vessel.

The means for maintaining the pH can include a pH probe for sensing the pH of the recirculating liquid, an inlet conduit for receiving make-up water into the sump, an overflow drain for preventing overfilling of the sump, and a control valve fluid connected in series with the fill conduit for blocking the inlet conduit in response to the pH probe when the pH reaches the high limit. Preferably the low limit is not less than 1.5 and the high limit is not greater than 4.0.

The system can also include means for receiving nutrients for the microorganisms into the liquid. The means for receiving nutrients can include a fill conduit for receiving fill water containing the nutrients into the recirculation system. The means for receiving the nutrients can further include an inlet conduit for receiving make-up water, a reservoir containing a concentration of the nutrients, and a feeder connected between the inlet conduit, the reservoir, and the fill conduit for mixing a dosage of the concentration of nutrients with the make up water to produce the fill water. Alternatively, the means for receiving the nutrients can include the tower vessel having an access structure for admitting a concentration of the nutrients into the vessel.

The nozzle can be one of a plurality of nozzles that are vertically oriented and horizontally spaced for evenly distributing the liquid downwardly onto the media. The nozzles are typically spaced not less than 10 feet above a lowermost media supporting surface of the media support structure so that the gas and the liquid each travel through 10 feet of the media. The tower vessel is preferably configured for directing the gas stream between the gas inlet and the exhaust outlet upwardly through the media, thereby producing counter-flow of the gas and the liquid.

The system can be provided in combination with the porous media, the porous media having a surface area of greater than 1000 times a corresponding cubic dimension of the media. Preferably the porous media has a surface area not less than approximately 10,000 times the cubic dimension for enhanced effectiveness in removing odor-carrying contaminants. Preferably the porous media comprises a concentration of an iron compound for enhanced effectiveness of the microorganisms. Most preferably, the porous media comprises lava rock.

Preferably the gas stream has a velocity of at least 50 feet per minute through the porous media and a static pressure drop of not more than 3.0 inches of water across a gas stream travel distance of approximately 10 feet through the porous media. The system can also include a fan for producing the gas flow between the gas inlet and the exhaust outlet. The tower vessel is preferably a fiberglass-reinforced plastic structure for high strength and corrosion resistance.

In another aspect of the invention, a process for removing contaminants including hydrogen sulfide from the incoming gas stream includes providing a porous media; populating the media with sulfur-oxidizing microorganisms; recirculating a liquid through the porous media; passing the gas stream through the porous media, to permit the microorganisms to oxidize the hydrogen sulfide to produce sulfuric acid; and maintaining a pH of the recirculating liquid between a low limit and a high limit, the low limit being not less than 1.0, the high limit being not greater than 5.0, thereby removing the hydrogen sulfide from the gas stream. The maintaining of the pH can include diluting the recirculating liquid with water, without requiring pH-balancing chemicals in the liquid. Preferably the pH low limit is not less than 1.5 and the high limit is not greater than 4.0. More preferably, the low limit is approximately 2.0 and the high limit is approximately 3.0.

In another aspect of the invention, a process for removing the contaminants includes providing porous media having a surface area of greater than 1000 times a corresponding cubic dimension of the media; populating the media with sulfur-oxidizing microorganisms; recirculating a liquid through the porous media; and passing the gas stream through the porous media, to permit the microorganisms to oxidize the hydrogen sulfide to produce sulfuric acid, thereby removing the hydrogen sulfide from the gas stream. Preferably the porous media has a surface area not less than approximately 10,000 times the cubic dimension. Preferably the porous media comprises lava rock. In the populating, the fill water can include primary effluent. The populating of the media can include receiving fill watercontaining the microorganisms into the recirculation system. The populating can further include receiving make-up water, and feeding the microorganisms from a reservoir into the make-up water to produce the fill water.

The process can further include receiving nutrients for the microorganisms into the liquid. The receiving of nutrients can include receiving fill water containing the nutrients into the recirculation system. The process can also include receiving make-up water, and filtering chlorine from the make-up water to form at least a portion of the fill water. The make-up water can be secondary effluent.

The receiving of the nutrients can also include receiving make-up water, and feeding the nutrients from a reservoir into the make-up water to produce the fill water. The feeding of the nutrients being into the make-up water having chlorine filtered therefrom. Alternatively, the receiving of the nutrients can include admitting a concentration of the nutrients onto the media.

The process can further include maintaining the pH of the recirculating liquid between the low and high limits, being not less than 1.0 and not greater than 5.0. Preferably the process further includes maintaining a flow rate of the recirculating liquid between approximately 1.5 gallons per minute and approximately 2.0 gallons per minute per square foot of plan area of the porous media. Also, the process preferably includes maintaining a gas stream velocity of at least 50 feet per minute through the porous media, with the gas stream having a static pressure drop of not more than 3.0 inches of water across a gas stream travel distance of approximately 10 feet through the porous media.

Thus the present invention provides effective, low-cost biological removal of contaminants including H₂S from air associated with wastewater treatment without adding other chemicals to the air stream.

### DRAWINGS

These and other features, aspects, and advantages of the present invention will become better understood with reference to the following description, and accompanying drawings, where:

FIG. 1 is an elevational view of an experimental prototype wastewater treatment system according to the present invention;

FIG. 2 is a graph of H₂S removal test results of the prototype system of FIG. 1;

FIG. 3 is a pictorial diagram of a preferred configuration of the system of FIG. 1;

FIG. 4 is an elevational view of a nozzle structure of the prototype system of FIG. 1;

FIG. 5 is an elevational view showing an alternative configuration of the system of FIG. 3;

FIG. 6 is a plan view of the system of FIG. 5.

### DESCRIPTION

The present invention is directed to a gas-liquid biotrickling scrubber system that is particularly effective in eliminating odors associated with wastewater treatment, especially the odor of hydrogen sulfide (H₂S). It has been discovered that a biotrickling scrubber constructed according the present invention and operating with gaseous and aqueous phases flowing, preferably in opposite directions, through a scrubber structure provides greater removal efficiencies at higher contaminate loadings than do biofilters. It has been further discovered that a suitable scrubbing solution can be recirculated directly, without additional treatment. Biotrickling scrubbers have smaller footprints than biofilters; they can be inoculated with specialized microorganisms, and they can be pH controlled, which is difficult to accomplish in biofilters.

### Pilot Plant Biotrickling Scrubber Testing

To determine if biotrickling scrubbers can be made to operate effectively for controlling hydrogen sulfide odors, large-scale field pilot plant research was conducted. Three biotrickling scrubbers were designed and tested using the following aerobic biological reaction:

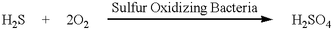

The pilot units were constructed with contactor columns made from fiberglass, having a circular plan cross-sectional shape and an internal diameter of 0.6 meters (2 feet). The units were 1.8 m (6 feet) in height, having a media bed depth of 1.2 m (4 feet). The temperature during the testing ranged from 10° C. (50° F.) to 35° C. (95° F.). Hydrogen sulfide concentrations typically ranged from as low as 1 to 2 ppm to over 300 ppm.

Three different types of packing configurations were tested to determine the most effective removal of H₂S at the lowest detention times. Scrubber “A” was constructed and was operated continuously for four years. Based on prior research experiments, a porous rock material was selected as the media. Due to initial concerns that this rock media could eventually break down and lead to plugging problems it was decided to also study the use of an engineered plastic packing media typically used in biotrickling filters for wastewater treatment. This second scrubber, known as Scrubber “B”, was in operation for over three months.

Due to the mediocre removals of H₂S that Scrubber “B” exhibited over the test period it was decided to change the packing. The third media (Scrubber “C”) evaluated was a special engineered plastic random-dump packing designed to have a large surface area, and a special surface to allow microorganisms to adhere to the plastic more effectively than standard plastic scrubber packing used in chemical scrubbers. This scrubber was operated for over eighteen months.

All three pilot plant packing materials were tested for sufficient time to determine how effectively they removed H₂S. Scrubber “A” was operated four years at empty bed detention times ranging from 12 to 15 seconds with inlet H₂S concentrations ranging from 4 to 380 ppm. Empty bed detention time is defined as the time required for the gas stream to travel through the space to be occupied by the media, without the media being present. With an average inlet concentration of 70 ppm Scrubber “A” achieved over 99% removal of H₂S. Scrubber “B” averaged 92% removal of inlet H₂S concentrations averaging 44 ppm with empty bed detention times of 20 seconds. During the eighteen months it was operated Scrubber “C” was able to remove 94% of the inlet H₂S, which averaged 54 ppm at the gas inlet when the detention times were set at approximately 20 seconds.

Although odor strength tests were not done on a regular basis, sampling for Scrubber “A” and “C” were conducted once a week for one month. Odor detectability was measured using dilution to threshold values (D/T) using odor panel olfactometry testing. The average inlet odor intensity for both pilot plants was approximately the same, at D/T values of about 20,000. Scrubber “A” was able to reduce inlet odors by 99%, but Scrubber “C” was not as successful, removing only 89%. No analyses were conducted for Scrubber “B”, but it was felt that odor removals would be similar to Scrubber “C”. It is believed that the packing of scrubber “C” had a surface area approaching, but not greater than approximately 500 square units per corresponding cubic unit (square feet per cubic foot).

### Full Scale Prototype Testing

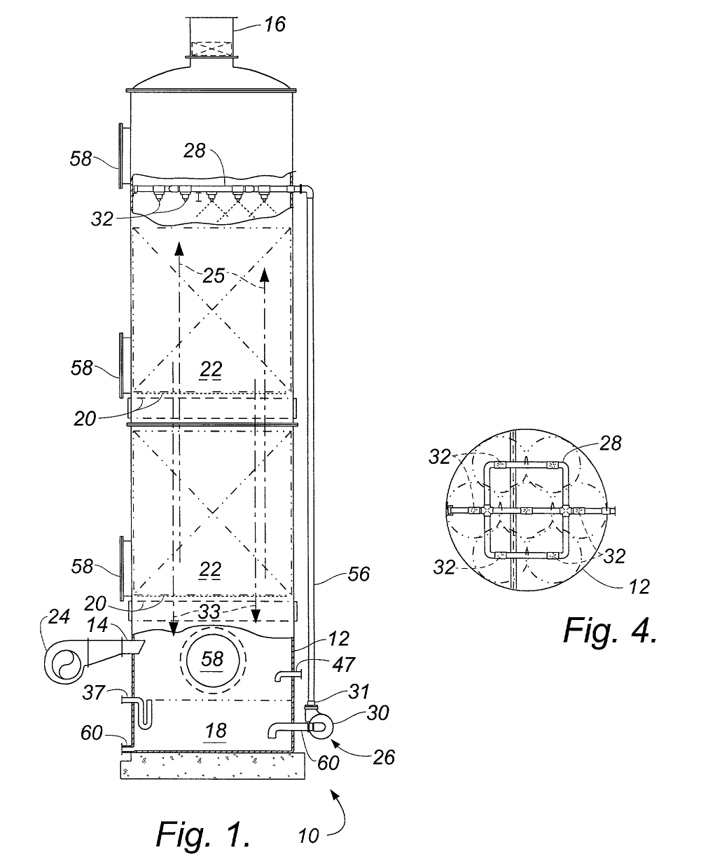

Based upon the results of pilot plant experiments, it was decided that construction of a full scale experimental scrubber system was warranted. This system, referred to herein as an experimental prototype, was designed to be large enough to replace a full-scale caustic scrubber unit, which was treating 42.5 m³/min (1500 ft³/min) of air. The pilot plant testing clearly showed that the porous rock media was the most effective packing media, and it was chosen to be used in the experimental prototype. Consequently, the full-sized experimental prototype was designed to hold porous rock media weighing approximately 9,000 kg (20,000 lbs.), to be operated in a seismically active area (meeting seismic zone 4 requirements), and being able to withstand 160 kilometer/min (100 mph) wind gusts. The experimental prototype included a scrubber tower configured as shown in FIG. 1 and described below.

The experimental prototype was placed at a location near a headworks that historically has had H₂S offgas concentrations ranging between 80 and 120 ppm. Since this was to be the first full-scale installation using the biotrickling method for removal of H₂S, the prototype was designed with a conservative empty bed detention time of 14 seconds. Once the scrubber tower was in place, the porous rock media was cleaned, treated, screened and installed. Specified properties of the media are a specific gravity of 1.65, 10 percent absorption (45.6 lbs. per cubic foot dry, 50.4 lbs. per cubic foot wet), a sediment height of zero, and a durability index of 100. Fiberglass ducting was run to the assembled prototype, and the sump of the scrubber tower was filled with 430 gallons of nutrient rich water. An additional 20 gallons of microorganisms were added to the prototype as “seed”, and the pH of the water was lowered to approximately 3.0 (to optimize bacterial growth).

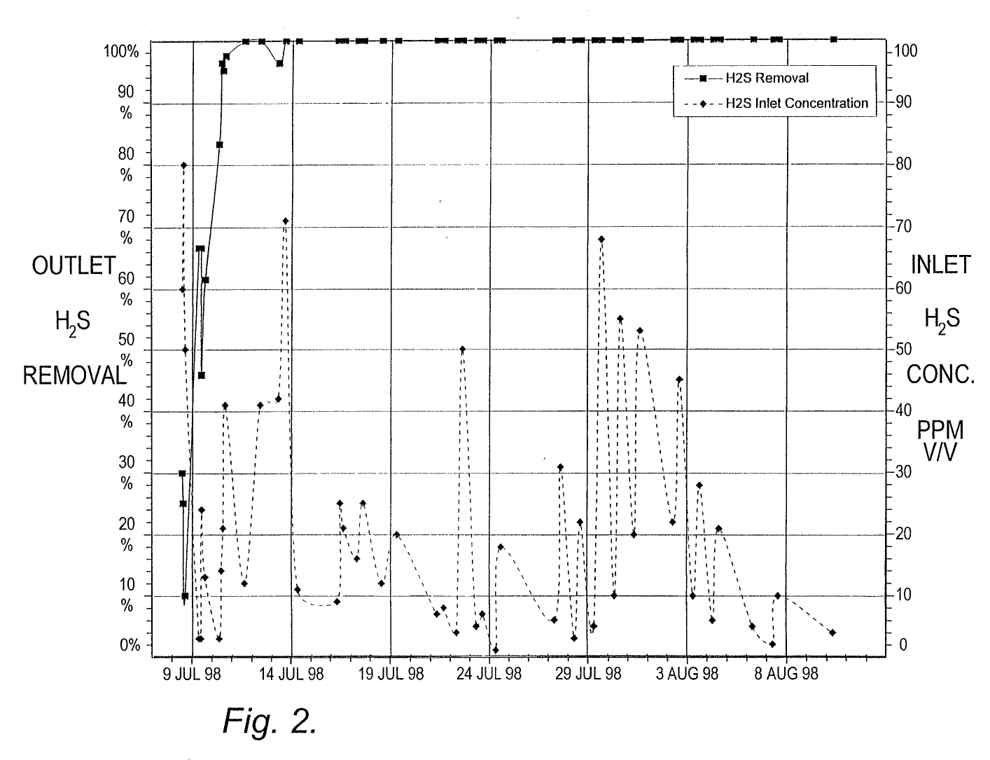

The nutrient rich water and microorganism “seed” were allowed to mix for several hours before the air to be treated was introduced to the tower. The first few measurements indicated that the experimental prototype was able to remove 30% of the H₂S. After 24 hours of operation the prototype was achieving removals of over 66%, and after 48 hours 98%. After 72 hours essentially 100% removal of H₂S was achieved, as can be seen in a plot of test results presented as FIG. 2.

When the experimental prototype was first started it operated with pressure losses across the media bed of approximately 5 cm (2 inches) of water column. After one month of operation the pressure losses increased to 6.4 cm (2.5 inches) of water column, and held at this level. One of the discoveries made was that the process biologically removed some of the organic compounds present in the air being treated; in particular, the aromatic VOC compounds. This is an important feature because the caustic scrubbers currently used do not remove any of these organic compounds. Activated carbon scrubbers remove organic compounds that pass through the wet caustic scrubber, with the removal efficiency of aromatic VOC's being the criteria for determining effective carbon life. Although the removals of aromatic compounds in the Bio-Scrubber are not extremely high, with the elimination of 40%-50% of the inlet concentrations, the Bio-Scrubber extended the life of the activated carbon unit down-stream by over ⅓ (33%) before change-out.

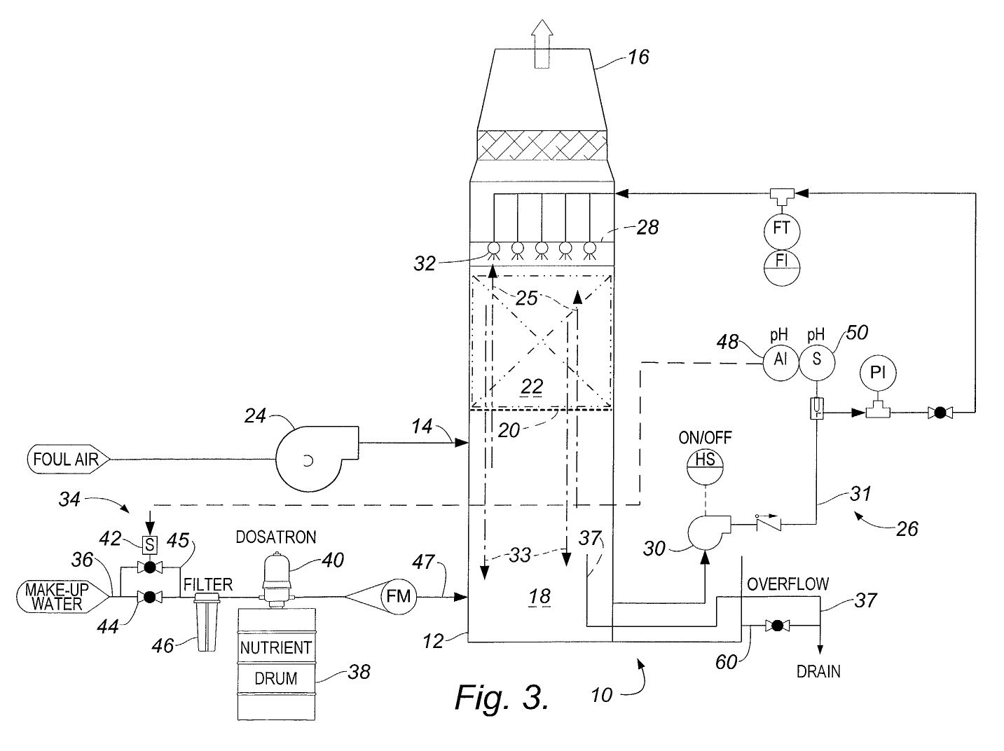

With reference to FIGS. 1, 3 and 4 of the drawings, an odor control scrubber system 10 according to the present invention includes a tower vessel 12 having an air inlet 14 and an exhaust outlet 16 spaced above the inlet, a lower portion of the vessel forming a sump 18 for a scrubbing solution. As shown in FIG. 1 and 3, a media support structure 20 extends over the sump 18 above the air inlet 14 for supporting porous media 22, and a fan 24 produces an upward gas flow stream 25 through the media 22 from the gas inlet 14 to the exhaust outlet 16. It will be understood that the gas flow stream 25 can be induced by external means; also, the fan 24 can be located anywhere in the path of the gas, including proximate or downstream of the exhaust outlet 18 as well as upstream of the gas inlet 16 as shown in the drawings. The system 10 also includes a recirculation system 26 having a nozzle structure 28 spaced above the media support structure 20, and a recirculation pump 30 connected in a recirculation line 31 between the sump 18 and a plurality of nozzles 32 of the nozzle structure 28 for producing a downward liquid flow stream 33 of scrubbing solution through the media 22. The downward flow stream 33 is induced by gravity, being preferably evenly distributed in the media 22. It will be further understood that although the gas flow stream 25 can be in any direction through the media 22, the upward direction providing preferred counter-flow orientations of the flow streams 25 and 29.

Further included in the scrubber system 10 is a liquid control system 34 for adding water (and optionally concentrations of sulfur-oxidizing microorganisms and/or nutrients) to the recirculating liquid. The liquid control system includes a make-up water inlet 36, a sump overflow outlet 37, and an optional concentrate reservoir 38 having a feeder unit 40 connected between the water inlet 36 and the tower vessel 12 for use when the supply of water into the water inlet 36 lacks a suitable concentration of nutrients. The liquid control system 34 also includes a control valve 42 and a manual bypass valve 44 fluid-connected in parallel between the make-up water inlet 36 and an inlet conduit 45, for controlling the flow of make-up water, an optional filter 46 being connected between the inlet conduit 45 and the feeder unit 40 for removing excessive amounts of chlorine that may be present in the make-up water. A fill conduit 47 is connected between the feeder unit 40 and the tower vessel 12 for passing the filtered make-up water together with concentrate dosages from the reservoir, into the sump 18. A pH monitor 48 operates the control valve 42 in response to a pH probe 50 that projects into the recirculating liquid as further described below. Preferably the pH probe is located in the recirculation line 31 as shown in FIG. 3. Alternatively, the probe extends into the sump 18. In either case, the probe 50 is preferably removable for calibration or inspection without shutdown of the scrubber system 10.

The recirculation line 31 includes a solution conduit 54 connected between the sump 18 and the pump 30, the nozzle structure 28 forming a nozzle manifold portion of the recirculation line 31 and supporting the nozzles 32 spaced above the media 22 in a vertically oriented and horizontally spaced apart array for evenly distributing the liquid onto the media, a feed conduit 56 being connected between the pump 30 and the nozzle structure 28.

As shown in FIGS. 1 and 3, the tower vessel 12 is provided with suitable hatches or manways 58 at appropriate locations for accessing the nozzle structure 28, the media 22, and the sump 18. The sump 18 has a drain fitting 60 as shown in FIG. 1. Also, appropriate anchor lugs and lift lugs (not shown) are formed on the vessel 12, which is preferably a fiberglass-reinforced-plastic (FRP) structure for high strength and corrosion resistance. As discussed above, FIG. 1 shows the configuration of the experimental prototype, the tower vessel 12 having an inside diameter of 6 feet and vertically spaced counterparts of the media support structure 20 for separately supporting 6.25-foot depths of the media 22 (12.5 feet total depth). A suitable form of the media support structure 20 is a grating having 2-inch square center spacing, constructed of FRP, and a plastic screen supported on the grating, the screen having a mesh spacing of approximately 0.25 inch.

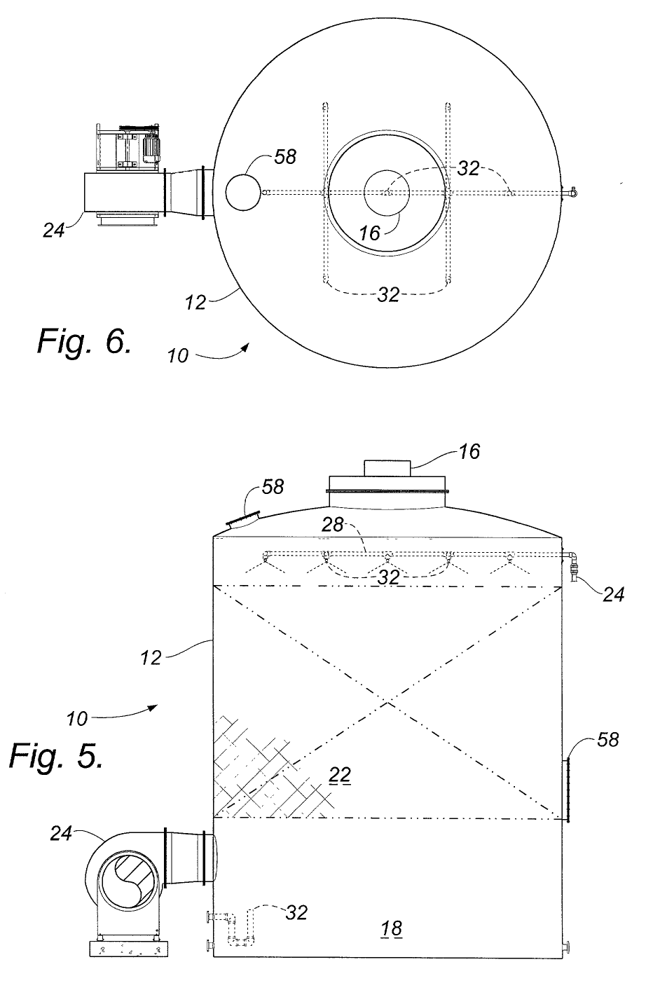

FIGS. 5 and 6 show portions of the scrubber system 10 altered in form. The tower vessel 12 of FIGS. 5 and 6 is configured to have an inside diameter of 18 feet, with space for the media 22 having a vertical depth of 11 feet. The fan 24 in this configuration is available as a HPCA 3000 fan, and the overflow outlet 37 is configured for the sump 18 to have a liquid level of 2 feet.

### Media

The porous media of the present invention serves as part of an ecosystem for the microorganisms. Typically there is 10 ft. of media as measured in the vertical plane. The media is chosen to eliminate treatment efficiency losses due to channeling and compacting. Porous rock media suitable for use as the media 22 is available from Global Environmental Solutions, Inc. (GESI), of Las Vegas, Nev. This media is made from lava rock, selected to have an exposed surface area of not less than 1000 square units per corresponding cubic unit, but more preferably approximately 10,000 square units per cubic unit. Plastic and solid organic media are regarded as unsuitable for use in the scrubber system 10 in that they have insufficient surface area, and they fail to support a uniformly high population of microorganisms over extended periods of time. The media 22 is cleaned and screened to an average size of approximately 1.5 inches, except that in tower vessels having an inside diameter of 4 feet or less the media is preferably sized approximately 1.0 inch. Other properties of the media 22 as supplied by GESI are as described above in connection with the experimental prototype. To prevent excessive damage during transit, the media 22 is preferably shipped independent of the tower vessel 12, being loaded after the vessel has been set in place and properly anchored.

### Start-Up and Testing

After installation, and verification of satisfactory airflow through the the scrubbing tower 12 operating conditions, the sump 18 is filled with make-up water and the pump 30, fan 24, piping, controls, and recirculation system 26 are checked for proper operation. Then the sump is “seeded” with the microorganisms, and the ecosystem is balanced and adjusted to optimize growth. The seeding can be by directly pouring a suitable concentrate into the sump*. More particularly, sulfuric acid can be added to the sump to lower the pH, preferably to approximately 3.0. When start-up occurs and untreated air is introduced, measurements should be taken of the levels of H₂S present in the air stream at the air inlet 14. After 48 hours measurements should again be taken at the air inlet and the exhaust outlet 16 to determine the level of growth of the microorganisms. After 72 hours further measurements should be taken, with continued monitoring twice per day until satisfactory performance is verified.

### Conclusions

Capital costs for construction of the scrubber system are expected to be higher than for traditional chemical scrubbers, because more detention time is required to remove H₂S, (dictating a larger unit), and the porous rock media requires stronger supporting structure than other systems. However, the scribber system 10 is believed to be much less expensive to operate than conventional chemical scrubbers. The cost to operate a caustic scrubber is currently estimated to be about $19.00 ft³/min of air treated. Full-scale operation of the scrubber system 10 is estimated to costs are about one-fifth, or $3.80 ft³/min of air treated. Results from testing both the pilot plants, and the full-scale experimental prototype indicate that the present invention will greatly reduce the chemical and labor costs required for odor control of wastewater treatment plant offgases. While it will be necessary in some cases to continue to maintain activated carbon scrubber as a polisher to remove odorous and other organic compounds, the scrubber system 10 substantially reduces the cost of operating activated carbon scrubbers by removing about half of incoming organic pollutants.

Although the present invention has been described in considerable detail with reference to contain preferred versions thereof, other versions are possible. Therefore, the spirit and scope of the appended claims should not necessarily be limited to the description of the preferred versions contained herein.

What is claimed is:

1. A gas-liquid scrubber system for removing contaminants including hydrogen sulfide from an incoming gas stream also containing oxygen, the system comprising:
(a) a tower vessel having a gas inlet for receiving the gas stream and an exhaust outlet, a perforate media support structure located between the gas inlet and the gas outlet for supporting porous media with the gas stream passing therethrough, and a sump for collecting liquid falling below the media support structure;
(b) a liquid recirculation system having a pump fluid connected to the sump, a nozzle in the tower vessel, and a conduit connected between the pump and the nozzle for spraying the media with the liquid when the media is supported on the media support structure and a quantity of the liquid is present in the sump, the liquid also passing through the media to the sump;
(c) means for populating the media with sulfur-oxidizing microorganisms; and
(d) means for maintaining a pH of the recirculating liquid between a low limit and a high limit, the low limit being not less than 1.0, the high limit being not greater than 5.0.

2. The system of claim 1, wherein the means for populating the media comprises a fill conduit for receiving fill water into the recirculation system, the fill water containing the microorganisms.

3. The system of claim 2, wherein the means for populating the media further comprises an inlet conduit for receiving make-up water, a reservoir containing a concentration of the microorganisms, and a feeder connected between the inlet conduit, the reservoir, and the fill conduit for mixing a dosage of the concentration of microorganisms with the make-up water to produce the fill water.

4. The system of claim 3, wherein the microorganisms comprise thiobacillus bacteria.

5. The system of claim 2, wherein the means for populating the media comprises the tower vessel having an access structure for admitting a concentration of the microorganisms into the vessel.

6. The system of claim 1, wherein the means for maintaining the pH comprises a pH probe for sensing the pH of the recirculating liquid, an inlet conduit for receiving make-up water into the sump, an overflow drain for preventing overfilling of the sump, and a control valve fluid connected in series with the fill conduit for blocking the inlet conduit in response to the pH probe when the pH reaches the high limit.

7. The system of claim 6, further wherein the low limit is not less than 1.5 and the high limit is not greater than 4.0.

8. The system of claim 1, further comprising means for receiving nutrients for the microorganisms into the liquid.

9. The system of claim 8, wherein the means for receiving nutrients comprises a fill conduit for receiving fill water into the recirculation system, the fill water containing the nutrients.

10. The system of claim 9, wherein the means for receiving the nutrients further comprises an inlet conduit for receiving make-up water, a reservoir containing a concentration of the nutrients, and a feeder connected between the inlet conduit, the reservoir, and the fill conduit for mixing a dosage of the concentration of nutrients with the make-up water to produce the fill water.

11. The system of claim 8, wherein the means for receiving the nutrients comprises the tower vessel having an access structure for admitting a concentration of the nutrients into the vessel.

12. The system of claim 1, wherein the nozzle is one of a plurality of nozzles, the nozzles being vertically oriented and horizontally spaced for evenly distributing the liquid downwardly onto the media.

13. The system of claim 12, wherein the nozzles are spaced not less than 10 feet above a lowermost media supporting surface of the media support structure.

14. The system of claim 1, wherein the tower vessel is configured for directing the gas stream between the gas inlet and the exhaust outlet upwardly through the media.

15. The system of claim 1, in combination with the porous media, the porous media having a surface area of greater than 1000 times a corresponding cubic dimension of the media.

16. The system of claim 15, wherein the porous media has a surface area not less than approximately 10,000 times the cubic dimension.

17. The system of claim 15, wherein the porous media comprises a concentration of an iron compound.

18. The system of claim 15, wherein the porous media comprises lava rock.

19. The system of claim 15, wherein the gas stream has a velocity of at least 50 feet per minute through the porous media and a static pressure drop of not more than 3.0 inches of water across a gas stream travel distance of approximately 10 feet through the porous media.

20. The system of claim 19, further comprising a fan for producing the gas flow between the gas inlet and the exhaust outlet.

21. The system of claim 1, further comprising a fan for producing the gas flow between the gas inlet and the exhaust outlet.

22. The system of claim 1, wherein the tower vessel is a fiberglass-reinforced plastic structure.

23. A process for removing contaminants including hydrogen sulfide from an incoming gas stream also containing oxygen, the process comprising:
(a) providing a porous media;
(b) populating the media with sulfur-oxidizing microorganisms;
(c) recirculating a liquid through the porous media;
(d) passing the gas stream through the porous media, to permit the microorganisms to oxidize the hydrogen sulfide to produce sulfuric acid; and
(e) maintaining a pH of the recirculating liquid between a low limit and a high limit, the low limit being not less than 1.0, the high limit being not greater than 5.0,
thereby removing the hydrogen sulfide from the gas stream.

24. The process of claim 23, wherein the maintaining the pH comprises diluting the recirculating liquid with water, without requiring pH-balancing chemicals in the liquid.

25. The process of claim 23, wherein the low limit is not less than 1.5 and the high limit is not greater than 4.0.

26. The process of claim 25, wherein the low limit is approximately 2.0 and the high limit is approximately 3.0.

27. A process for removing contaminants including hydrogen sulfide from an incoming gas stream also containing oxygen, the process comprising:
(a) providing a porous media having a surface area of greater than 1000 times a corresponding cubic dimension of the media;
(b) populating the media with sulfur-oxidizing microorganisms;
(c) recirculating a liquid through the porous media; and
(d) passing the gas stream through the porous media, to permit the microorganisms to oxidize the hydrogen sulfide to produce sulfuric acid,
thereby removing the hydrogen sulfide from the gas stream.

28. The process of claim 27, wherein the porous media has a surface area not less than approximately 10,000 times the cubic dimension.

29. The process of claim 27, wherein the porous media comprises lava rock.

30. The process of claim 27, wherein the populating the media comprises receiving fill water into the recirculation system, the fill water containing the microorganisms.

31. The process of claim 30, wherein in the populating, the fill water comprises primary effluent.

32. The process of claim 30, wherein the populating the media further comprises receiving make-up water, and feeding the microorganisms from a reservoir into the make-up water to produce the fill water.

33. The process of claim 27, further comprising receiving nutrients for the microorganisms into the liquid.

34. The process of claim 27, wherein the receiving the nutrients comprises receiving fill water into the recirculation system, the fill water containing the nutrients.

35. The process of claim 34, further comprising receiving make-up water, and filtering chlorine from the make-up water, the fill water comprising the make-up water having chlorine filtered therefrom.

36. The process of claim 35, wherein the make-up water is secondary effluent.

37. The process of claim 33, wherein the receiving the nutrients further comprises receiving make-up water, and feeding the nutrients from a reservoir into the make-up water to produce the fill water.

38. The process of claim 37, further comprising filtering chlorine from the make-up water, the feeding of the nutrients being into the filtered make-up water.

39. The process of claim 33, wherein the receiving the nutrients comprises admitting a concentration of the nutrients onto the media.

40. The process of claim 33, wherein the microorganisms comprise thiobacillus bacteria.

41. The process of claim 27, further comprising maintaining a pH of the recirculating liquid between a low limit and a high limit, the low limit being not less than 1.0, the high limit being not greater than 5.0.

42. The process of claim 27, further comprising maintaining a flow rate of the recirculating liquid between approximately 1.5 gallons per minute and approximately 2.0 gallons per minute per square foot of plan area of the porous media.

43. The process of claim 27, further comprising maintaining a gas stream velocity of at least 50 feet per minute through the porous media, with the gas stream having a static pressure drop of not more than 3.0 inches of water across a gas stream travel distance of approximately 10 feet through the porous media.
